(19) United States
(12) Patent Application Publication
(10) Pub. No.: US 2012/0209107 A1
(43) Pub. Date: Aug. 16, 2012

(54) METHOD AND APPARATUS FOR ENHANCING NEEDLE VISUALIZATION IN ULTRASOUND IMAGING

(75) Inventors: Jianjun Guo, Wuxi (CN); Zhi Xu, Wuxi (CN); Feng Lin, Wuxi (CN); Menachem Halmann, Bayside, WI (US); Mirsaid Seyed-Bolorforosh, Lake Forest, IL (US)

(73) Assignee: GENERAL ELECTRIC COMPANY, Schnectady, NY (US)

(21) Appl. No.: 13/337,713

(22) Filed: Dec. 27, 2011

(30) Foreign Application Priority Data
Dec. 27, 2010 (CN) 201010624654.3

(51) Int. Cl. A61B 8/13

(52) U.S. Cl. 600/424

(57) ABSTRACT

A method of enhancing needle visualization in ultrasound imaging is provided. The method includes reducing overall gain in needle frames, and applying a nonlinear mapping to the needle frames, wherein the nonlinear mapping is configured to make strong signals stronger and make weak signals weaker after mapping.

### CROSS REFERENCE TO RELATED APPLICATIONS

This application claims the benefit of Chinese Patent Application No. 201010624654.3 filed Dec. 27, 2010, which is hereby incorporated by reference in its entirety.

### BACKGROUND OF THE INVENTION

The present invention relates to an ultrasound imaging technique, and in particular, to a method and an apparatus for enhancing needle visualization in ultrasound imaging in image guidance applications.

Medical device ultrasound guidance is used in many medical applications for the purpose of guiding various types of invasive medical devices, such as aspiration and biopsy needles, etc., towards specific targets within a patient's body. The guidance can simplify such procedures and make them safer and quicker to perform. To visualize a needle most efficiently, a wide-angle scan frame orthogonal to needle direction is used to get the most echoes back from the needle. General methods such as B steer will introduce artifacts when compounding the wide-angle frame directly.

Therefore, there is a need for a method and an apparatus that can suppress artifacts and tissue signals while enhancing needle signals in the wide-angle frame before compounding the frame.

### SUMMARY OF THE INVENTION

A method and an apparatus that can suppress artifacts and tissue signals while enhancing needle signals are provided.

In one aspect, a method for enhancing needle visualization in ultrasound imaging is provided. The method includes an adjusting step for reducing overall gain in needle frames, and a mapping step for applying a nonlinear mapping to the needle frames, wherein the nonlinear mapping is arranged to make strong signals stronger and make weak signals weaker after mapping.

According to one embodiment, the method for enhancing needle visualization in ultrasound imaging may further include an enhancement step for applying edge enhancement filtering to the needle frames.

According to another embodiment, in the enhancement step, a filter template with non-zero coefficients along average needle directions is defined, and then cross-correlation is made between the needle frames and the filter template.

According to another embodiment, the method for enhancing needle visualization in ultrasound imaging may further include a thresholding step in which for each pixel point in each needle frame, if its correlation value is smaller than a predetermined threshold value, then the pixel point is set to zero.

According to another embodiment, the method for enhancing needle visualization in ultrasound imaging may further include a controlling step for applying time gain control to a uniform needle brightness along depth.

According to another embodiment, the method for enhancing needle visualization in ultrasound imaging may further include a configuration step for differently configuring transmitting frequency, receiving equalization filter, line density, and/or focal zone positions of the needle frames to obtain a needle image with minimal artifacts.

According to another embodiment, the method for enhancing needle visualization in ultrasound imaging may further include a filling step for using beams from the same origin but with different steering angles to fill a distribution loss area of the needle frame linear beams. In one embodiment, different gains are applied to the beams with different steering angles to compensate for different reflection effects of each beam from the needle.

In another aspect, a method for enhancing needle visualization in ultrasound imaging is provided. The method includes a filtering step for filtering a wide-angle image of a needle with an anisotropic filter to remove noise and preserve edge information, a detecting step for detecting image edge, a transforming step for applying Hough transform to the detected image, a determining step for determining parallel lines by using a result from the Hough transform and a generating step for generating a pattern matrix, and filling a area between the parallel lines with 1 and the rest with 0.

In yet another aspect, an apparatus for enhancing needle visualization in ultrasound imaging is provided. The apparatus includes an adjusting module for reducing overall gain in needle frames, and a mapping module for applying a nonlinear mapping to the needle frames, wherein the mapping module is configured to make strong signals stronger and make weak signals weaker after nonlinear mapping.

According to one embodiment, the apparatus for enhancing needle visualization in ultrasound imaging may further include an enhancement module for applying edge enhancement filtering to the needle frames.

According to another embodiment, the enhancement module is used to define a filter template with non-zero coefficients along average needle directions, and then to make cross-correlation between the needle frames and the filter template.

According to another embodiment, the apparatus for enhancing needle visualization in ultrasound imaging may further include a thresholding module that for each pixel point in each needle frame, sets the pixel point to zero if its correlation value is smaller than a predetermined threshold value.

According to another embodiment, the apparatus for enhancing needle visualization in ultrasound imaging may further include a controlling module for applying time gain control to a uniform needle brightness along depth.

According to another embodiment, the apparatus for enhancing needle visualization in ultrasound imaging may further include a configuration module for differently configuring transmitting frequency, receiving equalization filter, line density, and/or focal zone positions of the needle frames to enhance needle image with minimal artifacts.

According to another embodiment, the apparatus for enhancing needle visualization in ultrasound imaging may further include a filling module that uses beams from the same origin but with different steering angles to fill a distribution loss area of the needle frame linear beams.

In yet another aspect, an ultrasound imaging guidance system that includes an apparatus for enhancing needle visualization in ultrasound imaging is provided.

The method and apparatus described herein can effectively highlight needles with little degradation of image quality, but don't require extra hardware or hardware modification, and eventually help to improve workflow of needle guidance procedures.

Exemplary embodiments are described herein with reference to the drawings, in which the same or substantially the same parts are denoted with the same reference signs.

### BRIEF DESCRIPTION OF THE DRAWINGS

FIG. 1 is a schematic diagram of an exemplary ultrasound imaging process in which a method for enhancing needle visualization in ultrasound imaging is applied;

FIG. 2 is a schematic flowchart of the method for enhancing needle visualization in ultrasound imaging;

FIG. 3 is a schematic diagram illustrating the method for applying edge enhancement to a needle frame;

FIG. 4 is a schematic diagram of nonlinear mapping;

FIG. 5 is a schematic diagram of a distribution of linear beams for tissue frames;

FIG. 6 is a schematic diagram of a distribution of linear beams for needle frames;

FIG. 7 is a schematic diagram illustrating loss of needle information after compounding of the image obtained by using linear beams;

FIG. 8 is a schematic diagram of using beams from the same origin but with different steering angles to fill a loss area;

FIG. 9 is a schematic diagram of a compounding frame without loss of needle information;

FIG. 10 is a schematic flowchart of an alternative method for enhancing needle visualization in ultrasound imaging;

FIG. 11 is a schematic diagram of an apparatus for enhancing needle visualization in ultrasound imaging;

FIG. 12 is a schematic diagram of an apparatus for enhancing needle visualization in ultrasound imaging.

### DETAILED DESCRIPTION OF THE INVENTION

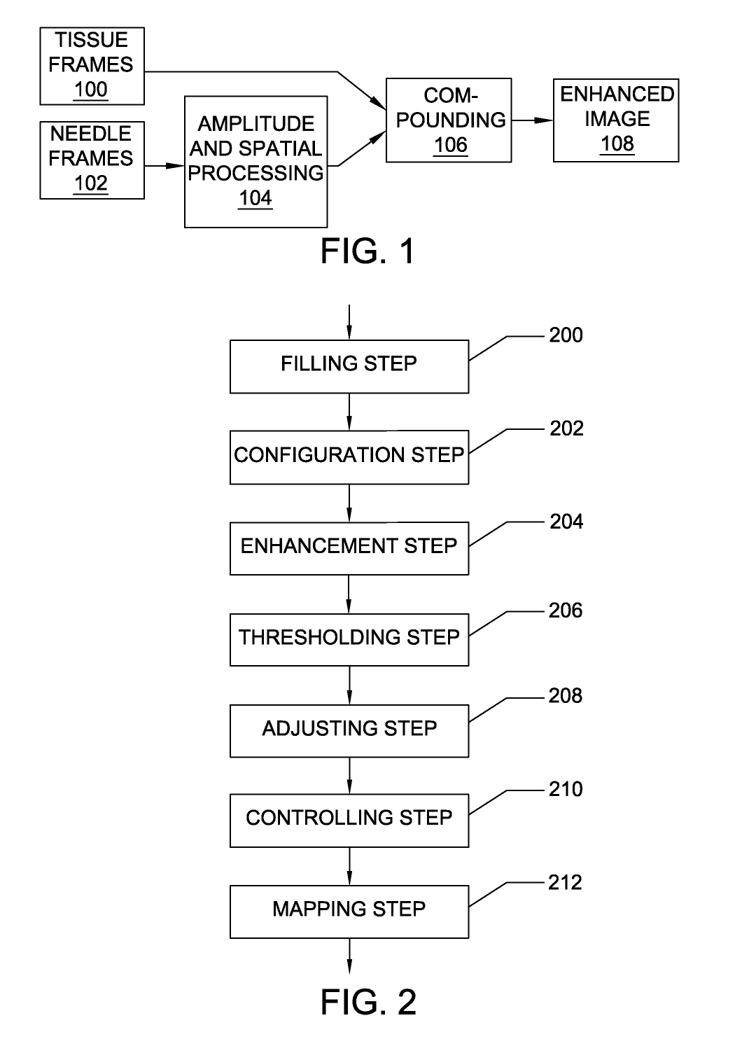

As a highly reflective object, a needle is highlighted in an ultrasound image when a scanning angle is vertical to or approximately vertical to the needle angle. However, it is very difficult to distinguish the needle from normal tissues in post-processing, because the image data is typically compressed to 8-bit. If this needle frame is compounded with the frames of normal tissues directly, it introduces artifacts produced by grating lobes and degraded tissue images together with strong needle signals. Therefore, it is critical to suppress artifacts and tissue signals while enhancing needle signals in wide-angle frames before compounding. As shown in FIG. 1, an exemplary method performs amplitude and spatial processing 104 on the needle frames 102 before compounding 106 the tissue frames 100 and the needle frames 102, and finally may obtain an image 108 with enhanced needle visualization.

FIG. 2 is a flowchart of an exemplary method for enhancing needle visualization in ultrasound imaging. The method includes an adjusting step 208 and a mapping step 212. In some embodiments, the method includes a filling step 200, a configuration step 202, an enhancement step 204, a thresholding step 206, and/or a controlling step 210. These steps are explained in details hereinafter.

One or more frames of tissue data and one or more frames of needle data are collected. Tissue frames can be configured for best tissue image quality. The tissue frame(s) may consist of only a straight frame like typical b-mode imaging, or multiple frames like what is typically implemented in spatial compounding, for example frames with steering angles of −15 degrees, 0 degree, and 15 degrees.

A needle frame is a frame with a large steering angle so that the beam direction is vertical or approximately vertical with respect to the needle direction. For example, the steering angle may be 45 degrees. At the same time, needle frame also may be a compounded frame of multi-angle frames. Angles at which a doctor inserts a needle are different in different applications. When the beam incidence direction for the needle frame is vertical to the needle direction, the best effect can be achieved. Thus, by means of multi-angle scanning, the needle frame can be obtained by compounding. For example, two frames resulting from 25 degree and 45 degree scanning can be directly compounded into a needle frame. The needle frame can be configured differently to maximally enhance needle image with minimal artifacts in configuration step 202. The different configurations may include transmitting frequency, receiving equalization filter, line density, focal zone positions, etc. For better image quality and less resources, the needle frame imaging may be configured differently from the tissue frames. For example, the imaging frequency is lowered to improve transmit element directivity, thereby improving image quality while suppressing grating lobes. This may be implemented by transmitting a low frequency waveform or reducing the center frequency of a receiving band-pass filter. Alternatively, harmonic imaging may be used to reduce grating lobes. Other configurations may be changed as well. For example, the number of transmitting focal zones may be reduced for better frame rate. Furthermore, since frequency is lowered, the number of beams used to construct a b-mode frame may be reduced to further improve the frame rate.

Furthermore, the needle frame can be further processed to enhance the needle and suppress the tissue and artifacts. The method according to one embodiment can process the needle frame based on amplitude information, spatial information or the combination of both. Amplitude processing may include gain adjustment, amplifying, thresholding, and nonlinear mapping, etc. Spatial processing may include anisotropic smoothing, edge enhancement by cross-correlating to a template, etc. The processed needle frames are then compounded with tissue frames to form the final enhanced image. The compounding may be implemented by using arithmetic averaging, maximal detection, etc.

In one embodiment, tissue frames are composed of frames of three regular steering angles, i.e., −15 degrees, 0 degree, and 15 degrees. A needle frame has a steering angle of 45 degrees, or a plurality of wide-angle scanning frames are compounded to obtain the needle frame.

Edge enhancement step 204 applies edge enhancement filtering to the needle frame. The edge enhancement method according to one embodiment is as follows. A template with non-zero coefficients along average needle directions is defined. Cross-correlation is made between the needle frame and the template. As a result, the needle is enhanced while other tissue signals and artifacts are suppressed.

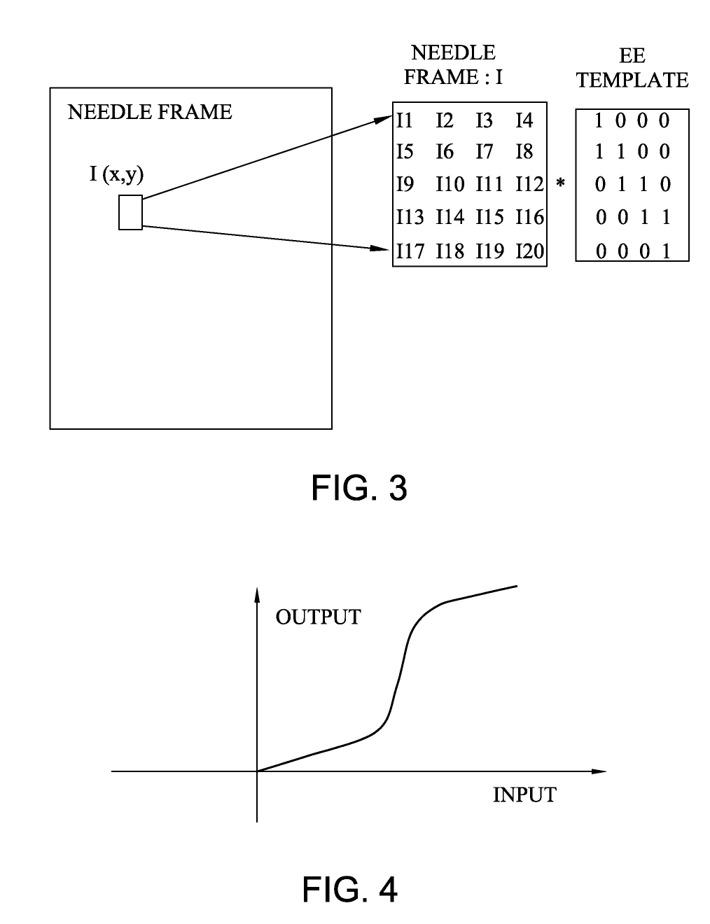

In addition, thresholding step 206 may apply thresholding to the resultant data to further suppress non-needle signals. As shown in FIG. 3, convolution is calculated between the EE filter template and the needle frame. For each point I (x, y) with coordinates (x, y) in the needle frame, if its correlation value is smaller than a predetermined threshold value, i.e., sum(I(i)*EE(i))/sum(I(i))<threshold, I (x, y) is set to zero, wherein the predetermined threshold value is an empiric value that can be determined through experimentation, in the interval [0, 1] In one embodiment, the predetermined threshold value is 0.4.

The overall gain in needle frames may also be adjusted in adjusting step 208. In other words, overall gain is reduced in needle data. Since needle signal is very strong in the needle frame, reducing gain further suppresses tissue/artifacts while maintaining good signal strength of the needle. Optionally, time gain control TGC may be applied for a uniform needle brightness along depth in controlling step 210.

Next, a nonlinear mapping may be applied so as to enhance the needle while suppressing tissue/artifacts in mapping step 212. The nonlinear mapping is designed so that strong signals (typically from the needle) are stronger while weak signals (typically from tissue or artifacts) are weaker. As shown in FIG. 4, the nonlinear mapping is a curved line along which the corresponding output value of each point in the needle frame can be found, and the transformed image would suppress weak signals while enhancing strong signals.

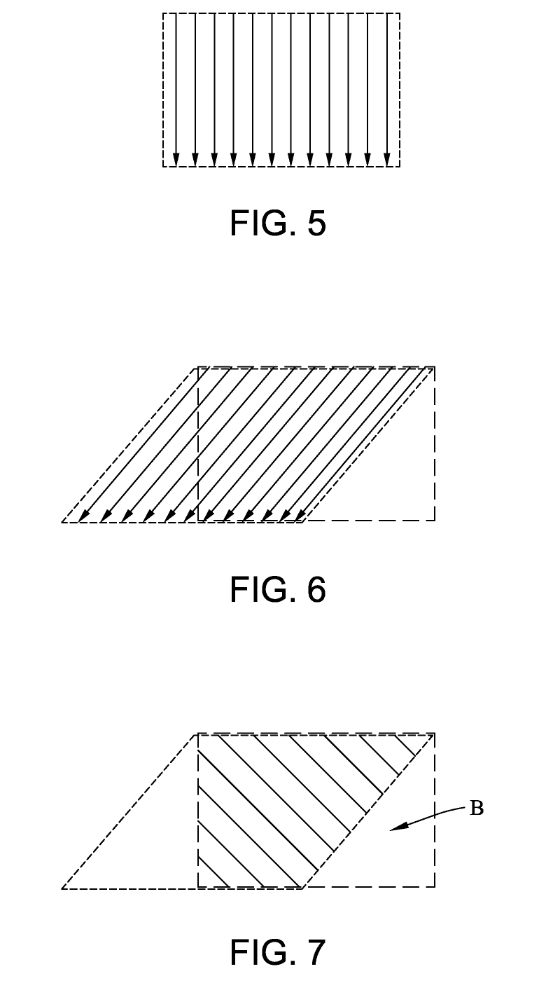

In addition, beam patterns of the needle frame and the tissue frame may also be different. The traditional tissue frame and needle frame both use linear beam distribution, as shown in FIGS. 5 and 6. This may lead to loss of needle information in a triangle region in the compounded image, indicated as region B in FIG. 7.

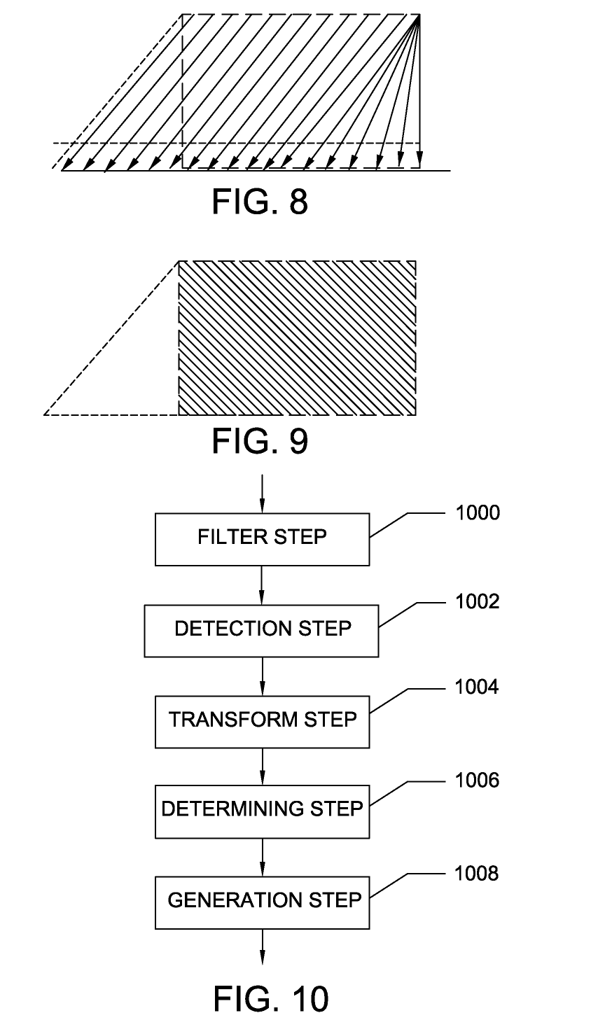

As shown in FIG. 8, in one embodiment, such a distribution of needle frame beams may be used, i.e., filling the triangle region with beams having the same origin but different steering angles in filling step 200. As the steering angle becomes smaller, the needle reflection can weaken gradually. It provides a better effect over the traditional configuration of abrupt cutoff.

In the needle frame, as the steering angles of scanning beams become smaller, the amplitude of the needle signals decreases accordingly. Thus, beams with different steering angles use different gains to compensate for different reflection effects of each beam from the needle. Therefore, a more consistent needle image can be obtained, as shown in FIG. 9.

According to another embodiment, an alternative method to maintain needle signal while suppressing tissue/artifacts in the needle frame is to use pattern recognition methods to identify needle region. Then, a mask may be generated so that data outside the needle region is 0. An example implementation is as follows.

As shown in FIG. 10, firstly, the wide-angle image of the needle is filtered by an anisotropic filter in a filter step 1000, where the anisotropic filter can remove noise and preserve edge information. Then, image edge detection is performed in a detection step 1002. Next, a Hough transform is performed on the detected image in a transform step 1004. Next, parallel lines are determined in a determining step 1006 by using the result from the Hough transform. Next, a pattern matrix is generated in a generation step 1008, and the area between parallel lines is filled with 1 and the rest is filled with 0. Then, the pattern matrix is multiplied by the original image. Finally, the resultant product may be compounded to the normal scanning sequence.

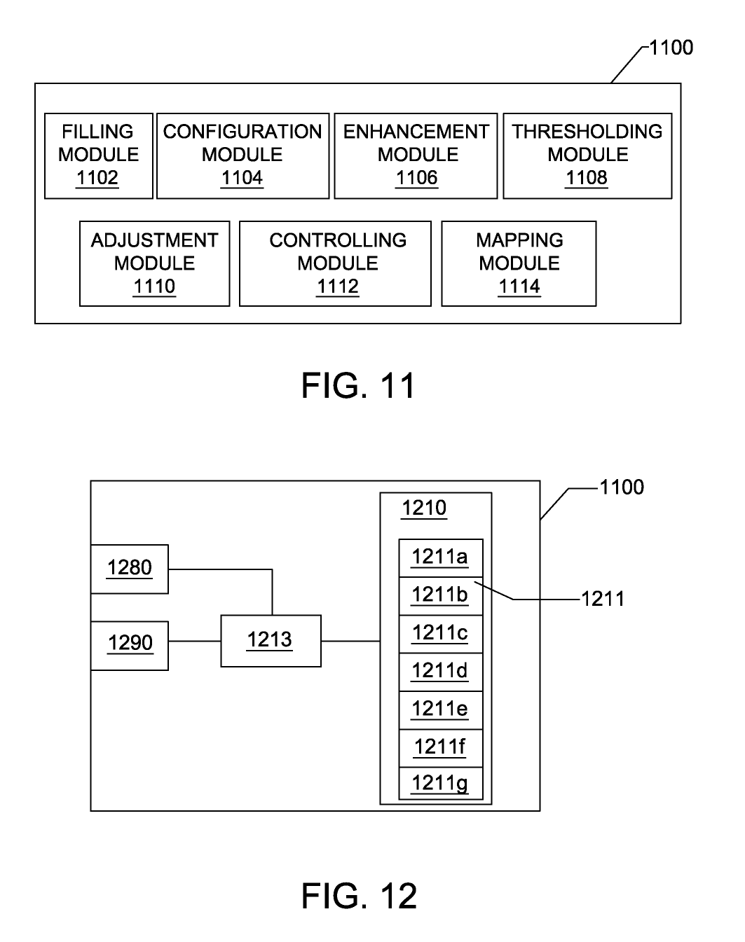

FIG. 11 is a schematic diagram of an apparatus 1100 for enhancing needle visualization in ultrasound imaging according to one embodiment. The apparatus 1100 includes an adjusting module 1110 and a mapping module 1114. In some embodiments, the apparatus 1100 may further include a filling module 1102, a configuration module 1104, an enhancement module 1106, a thresholding module 1108, and/or a controlling module 1112. These modules may be implemented by means of software, hardware, firmware or any combination thereof. The filling module 1102 is used to perform the step 200, the configuration module 1104 is used to perform the step 202, the enhancement module 1106 is used to perform the step 204, the thresholding module 1108 is used to perform the step 206, the adjusting module 1110 is used to perform the step 208, the controlling module 1112 is used to perform the step 210, and the mapping module 1114 is used to perform the step 212.

FIG. 12 shows another embodiment of the apparatus 1100 for enhancing needle visualization in ultrasound imaging. The apparatus 1100 includes a processing unit 1213, for example, MCU, DSP or CPU, etc. The processing unit 1213 may be a single unit or a plurality of units for performing the different steps. In addition, the apparatus 1100 further may include an interaction interface 1280 and an output unit 1290 for inputting the collected needle image data and outputting the processed needle image data. In addition, the apparatus 1100 further may include at least one computer program product 1210 in the form of non-volatile memory, for example, EEPROM, flash memory, or hard disk drive, etc. The computer program product 1210 includes a computer program 1211 including program codes which, when being executed, cause the apparatus 1100 to perform the steps shown in FIG. 2.

Specifically, the program codes in the computer program 1211 for the apparatus 1100 include a filling module 1211a for performing the step 200, a configuration module 1211b for performing the step 202, an enhancement module 1211c for performing the step 204, a thresholding module 1211d for performing the step 206, an adjusting module 1211e for performing the step 208, a controlling module 1211f for performing the step 210, and a mapping module 1211g for performing the step 212. In other words, when the different modules 1211a-1211g are executed on the processing unit 1213, they correspond to the modules 1102, 1104, 1106, 1108, 1110, 1112 and 1114 shown in FIG. 11.

The apparatus 1100 for enhancing needle visualization in ultrasound imaging according to the above embodiments may be implemented in various ultrasound imaging guidance systems by means of software, hardware, firmware or any combination thereof. The implementation is easy for persons skilled in the art, and accordingly, is not described in detail herein.

While the present invention has been described with reference to specific exemplary embodiments, it is not confined to these specific embodiments. Persons skilled in the art should understand that various modifications, replacements, changes and the like may be made to the invention. For example, one step or module in the above embodiments can be divided into two or more steps or modules. Further, two or more steps or modules in the above embodiments can be incorporated into one step or module. However, all of these fall within the scope of the present invention, without departing from the spirit of the invention. In addition, terms used herein are not limitations, but only serve the purpose of illustration. Furthermore, “one embodiment”, “another embodiment” and the like as used herein may refer to different embodiments. Certainly, all of these embodiments or at least some of them may be combined in one embodiment.

1. A method of enhancing needle visualization in ultrasound imaging, comprising:
reducing overall gain in needle frames; and
applying a nonlinear mapping to the needle frames,
wherein the nonlinear mapping is configured to make strong signals stronger and make weak signals weaker after mapping.

2. The method of enhancing needle visualization in ultrasound imaging according to claim 1, wherein the method further comprises:
applying edge enhancement filtering to the needle frames.

3. The method of enhancing needle visualization in ultrasound imaging according to claim 2, wherein applying edge enhancement filtering comprises:
defining a filter template with non-zero coefficients along average needle directions; and
cross-correlating the needle frames and the filter template.

4. The method of enhancing needle visualization in ultrasound imaging according to claim 3, wherein the method further
comprises setting each pixel point in each needle frame that has a correlation value smaller than a predetermined threshold value equal to zero.

5. The method of enhancing needle visualization in ultrasound imaging according to claim 1, wherein the method further
comprises applying time gain control to a uniform needle brightness along a depth.

6. The method of enhancing needle visualization in ultrasound imaging according to claim 1, wherein the method further comprises differently configuring at least one of transmitting frequency, receiving equalization filter, line density, and focal zone positions of the needle frames such that the needle image is enhanced with minimal artifacts.

7. The method of enhancing needle visualization in ultrasound imaging according to claim 1, wherein the method further comprises using beams with the same origin but with different steering angles to fill a distribution loss area of needle frame linear beams.

8. The method of enhancing needle visualization in ultrasound imaging according to claim 7, wherein using beams comprises applying different gains to beams with different steering angles to compensate for different reflection effects of each beam from the needle.

9. A method of enhancing needle visualization in ultrasound imaging, comprising:
filtering a wide-angle image of a needle with an anisotropic filter to remove noise and preserve edge information;
detecting an image edge;
applying a Hough transform to the detected image;
determining parallel lines using a result of the Hough transform; and
generating a pattern matrix; and
filling an area between the parallel lines with 1; and
filling any remaining areas with 0.

10. An apparatus for enhancing needle visualization in ultrasound imaging, comprising:
an adjusting module configured to reduce overall gain in needle frames; and
a mapping module configured to apply a nonlinear mapping to the needle frames, wherein the mapping module is configured to make strong signals stronger and make weak signals weaker after nonlinear mapping.

11. The apparatus for enhancing needle visualization in ultrasound imaging according to claim 10, wherein the apparatus further comprises an enhancement module applying configured to apply edge enhancement filtering to the needle frames.

12. The apparatus for enhancing needle visualization in ultrasound imaging according to claim 11, wherein the enhancement module is further configured to define a filter template with non-zero coefficients along average needle directions, and to cross-correlate between the needle frames and the filter template.

13. The apparatus for enhancing needle visualization in ultrasound imaging according to claim 12, wherein the apparatus further comprises a thresholding module configured to set each pixel point in each needle frame that has a correlation value smaller than a predetermined threshold value equal to zero.

14. The apparatus for enhancing needle visualization in ultrasound imaging according to claim 10, wherein the apparatus further comprises a controlling module configured to apply time gain control to a uniform needle brightness along a depth.

15. The apparatus for enhancing needle visualization in ultrasound imaging according to claim 10, wherein the apparatus further comprises a configuration module configured to differently configure at least one of transmitting frequency, receiving equalization filter, line density, and focal zone positions of the needle frames to obtain a needle image with minimal artifacts.

16. The apparatus for enhancing needle visualization in ultrasound imaging according to claim 10, wherein the apparatus further comprises a filling module configured to use beams with the same origin but with different steering angles to fill a distribution loss area of needle frame linear beams.

17. An ultrasound imaging guidance system comprising the apparatus for enhancing needle visualization in ultrasound imaging according to claim 10.

18. The ultrasound imaging guidance system according to claim 17, wherein the apparatus further comprises an enhancement module configured to apply edge enhancement filtering to the needle frames.

19. The ultrasound imaging guidance system according to claim 18, wherein the enhancement module is further configured to define a filter template with non-zero coefficients along average needle directions, and to cross-correlate between the needle frames and the filter template.

20. The ultrasound imaging guidance system according to claim 19, wherein the apparatus further comprises a thresholding module configured to set each pixel point in each needle frame that has a correlation value smaller than a predetermined threshold value equal to zero.
